(19) United States
(12) Patent Application Publication
(10) Pub. No.: US 2022/0288287 A1
(43) Pub. Date: Sep. 15, 2022

(54) SYSTEM AND METHOD FOR CASSETTE IDENTIFICATION AND LOCKOUT

(71) Applicant: Johnson & Johnson Surgical Vision, Inc., Santa Ana, CA (US)

(72) Inventor: Scott G. Strayer, Rancho Santa Margarita, CA (US)

(21) Appl. No.: 17/202,291

(22) Filed: Mar. 15, 2021

(51) Int. Cl. A61M 1/00; A61F 9/007

(52) U.S. Cl. CPC A61M 1/0058; A61F 9/00745; A61M 2205/12; A61M 2205/14; A61M 2205/3334; A61M 2210/0612

(57) ABSTRACT

The present invention is generally directed to improved methods, devices, and systems for controlling the use of surgical cassette in a surgical system, comprising a console, a handpiece in communication with the console, a cassette receptacle configured to couple a cassette to the console having a pre-load detection system, and a cassette having a body portion configured to contact the pre-load detection system, wherein an indication from the pre-load detection system identifies the cassette, and wherein the system adjusts one or more settings based on the identified cassette.

### TECHNICAL FIELD OF THE INVENTION

The present invention is generally related to methods, devices, and systems for controlling and identifying the use of surgical cassettes, particularly during treatment of an eye.

### BACKGROUND OF THE INVENTION

The optical elements of the eye include both a cornea (at the front of the eye) and a lens within the eye. The lens and cornea work together to focus light onto the retina at the back of the eye. The lens also changes in shape, adjusting the focus of the eye to vary between viewing near objects and far objects. The lens is found just behind the pupil, and within a capsular bag. This capsular bag is a thin, relatively delicate structure which separates the eye into anterior and posterior chambers.

With age, clouding of the lens, or cataracts, are fairly common. Cataracts may form in the hard central nucleus of the lens, in the softer peripheral cortical portion of the lens, or at the back of the lens near the capsular bag.

Cataracts can be treated by the replacement of the cloudy lens with an artificial lens. Phacoemulsification systems often use ultrasound energy to fragment the lens and aspirate the lens material from within the capsular bag. This may allow the capsular bag to be used for positioning of the artificial lens, and maintains the separation between the anterior portion of the eye and the vitreous humour in the posterior chamber of the eye.

During cataract surgery and other therapies of the eye, accurate control over the volume of fluid within the eye is highly beneficial. For example, while ultrasound energy breaks up the lens and allows it to be drawn into a treatment probe with an aspiration flow, a corresponding irrigation flow may be introduced into the eye so that the total volume of fluid in the eye does not change excessively. If the total volume of fluid in the eye is allowed to get too low at any time during the procedure, the eye may collapse and cause significant tissue damage. Similarly, excessive pressure within the eye may strain and injure tissues of the eye.

While a variety of specific fluid transport mechanisms have been used in phacoemulsification and other treatment systems for the eyes, aspiration flow systems can generally be classified in two categories: 1) volumetric-based aspiration flow systems using positive displacement pumps; and 2) vacuum-based aspiration systems using a vacuum source, typically applied to the aspiration flow through an air-liquid interface. These two categories of aspiration flow systems each have unique characteristics that render one more suitable for some procedures than the other, and vice versa.

Among positive displacement aspiration systems, peristaltic pumps (which use rotating rollers that press against a flexible tubing to induce flow) are commonly employed. Such pumps provide accurate control over the flow volume. The pressure of the flow, however, is less accurately controlled and the variations in vacuum may result in the feel or traction of the handpiece varying during a procedure. Peristaltic and other displacement pump systems may also be somewhat slow.

Vacuum-based aspiration systems provide accurate control over the fluid pressure within the eye, particularly when combined with gravity-fed irrigation systems. While vacuum-based systems can result in excessive fluid flows in some circumstances, they provide advantages, for example, when removing a relatively large quantity of the viscous vitreous humour from the posterior chamber of the eye. However, Venturi pumps and other vacuum-based aspiration flow systems are subject to pressure surges during occlusion of the treatment probe, and such pressure surges may decrease the surgeon's control over the eye treatment procedure.

Different tissues may be aspirated from the anterior chamber of the eye with the two different types of aspiration flow. For example, vacuum-induced aspiration flow may quickly aspirate tissues at a significant distance from a delicate structure of the eye (such as the capsular bag), while tissues that are closer to the capsular bag are aspirated more methodically using displacement-induced flows.

Conventionally, fluid aspiration systems include a console and a fluidic cassette mounted on the console. The fluidic cassette is typically changed for each patient and cooperates with the console to provide fluid aspiration. Generally, a single type of cassette is used by a particular console, regardless of whether the procedure will require positive displacement aspiration, vacuum-based aspiration, or both. U.S. Pat. Nos. 8,070,712 and 9,295,765 provide examples of cassettes currently used in the marketplace, the contents of each are herewith incorporated by reference in their entirety as if set forth herein.

With some surgical systems in the art, cassettes having the same size and shape may be forward or backwards compatible with different system and may have different functionality associated with each. Since attributes and functionality may vary between cassettes that share the same standard size and shape there needs to be a mechanism to ensure only cassettes compatible with a system are used and the system operates correctly based on the type of compatible cassette is used to avoid unintended operational failures.

In light of the above, it would be advantageous to provide improved devices, systems, and methods for the identification of surgical cassettes.

### SUMMARY OF THE INVENTION

The present invention provides for an eye surgical system, comprising a cassette having at least two body portions and a console, wherein the console comprises a cassette receptacle configured to couple the cassette with the console, the cassette receptacle comprises a cassette preload detection system, the body portions of the cassette are configured to contact the cassette pre-load detection system, and wherein the configuration of the body portions identifies a type of cassette coupled with the console. The cassette pre-load detection system may comprise two detection switches and the two detection switches may be proximate to each other. Each type of cassette may correspond to a console compliant attribute selected from the group consisting of tubing diameter, tubing material, tubing thickness, tubing length, vacuum pressure limit, aspiration pressure limit, fluid flow rate; and irrigation rate. The body portion of the cassette may be configured to contact one of two detection switches which may be proximate to the top of the cassette body and may comprise a microswitch, piezometer, or an optical switch. The console may receive an indication of the depth of one of the body portions of a cassette from one of the detection switches and may receive an indication of a body portion from one of the detection switches. The console may also operate in accordance with the cassette type identified.

The present invention provides for a surgical console configured to receive multiple types of eye treatment cassettes, comprising a cassette receptacle for interchangeably receiving multiple types of cassettes, wherein the console operates with a first type of cassette and does not operate with a second type of cassette, and a console controller for receiving an indication of the type of cassette received by the cassette receptacle and controlling the operation of the console. A cassette type indication may be generated by a cassette pre-load detection system and the indication received may be from a detection switch. The indication may be indicative of the depth of a body portion of the first type of cassette or second type of cassette.

The present invention provides a method for identifying multiple types of eye treatment cassettes, comprising interchangeably receiving the multiple types of eye treatment cassettes in a cassette receptacle of a surgical console, enabling operation of the console when a first type of cassette is received, and receiving an indication of the first type of cassette from one of at least two pre-load detection switches. The second type of cassette may be configured to contact each of the two pre-load detection switches and the pre-load detection switches may comprise a microswitch or an optical switch, for example.

### BRIEF DESCRIPTION OF THE DRAWINGS

The present Invention is best understood with reference to the following detailed description of the invention and the drawings in which:

FIG. 1 schematically illustrates an eye treatment system in which a cassette couples an eye treatment probe with a surgical console;

FIGS. 2A and 2B illustrate an exemplary surgical cassette for use in the system of FIG. 1;

FIGS. 3A and 3B are front views of an exemplary surgical cassette receptacle of a surgical console configured to receive multiple types of eye treatment cassettes; and

FIGS. 4A-4C are a top view of an exemplary surgical cassette.

### DETAILED DESCRIPTION OF THE INVENTION

Reference will now be made in detail to embodiments of the invention, examples of which are illustrated in the accompanying drawings. While the invention will be described in conjunction with the embodiments, it will be understood that they are not intended to limit the invention to those embodiments. On the contrary, the Invention is intended to cover alternatives, modifications, and equivalents, which may be included within the spirit and scope of the invention as defined by the appended claims.

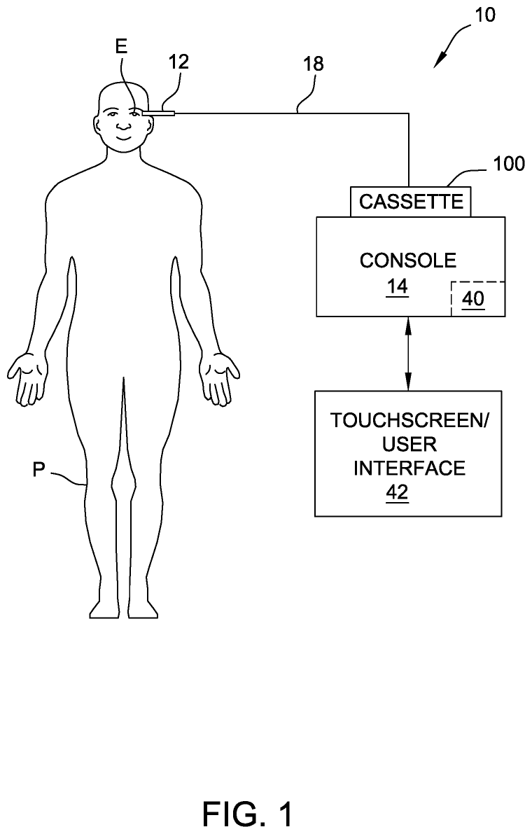

Referring to FIG. 1, a system 10 for treating an eye E of a patient P generally includes an eye treatment probe handpiece 12 coupled to a console 14 by a cassette 100 mounted on the console, Handpiece 12 may include a handle for manually manipulating and supporting an insertable probe tip. The probe tip has a distal end which is insertable into the eye, with one or more lumens in the probe tip allowing irrigation fluid to flow from the console 14 and/or cassette 100 into the eye. Aspiration fluid may also be withdrawn through a lumen of the probe tip, with the console 14 and cassette 100 generally including a vacuum aspiration source, a positive displacement aspiration pump, or both to help withdraw and control a flow of surgical fluids into and out of eye E. As the surgical fluids may include biological materials that should not be transferred between patients, cassette 100 will often comprise a disposable (or alternatively, sterilizable) structure, with the surgical fluids being transmitted through flexible conduits 18 of the cassette that avoid direct contact in between those fluids and the components of console 14.

When a distal end of the probe tip of handpiece 12 is inserted into an eye E for example, for removal of a lens of a patient with cataracts, an electrical conductor and/or pneumatic line (not shown) may supply energy from console 14 to an ultrasound transmitter of the handpiece, a cutter mechanism, or the like. Alternatively, the handpiece 12 may be configured as an irrigation/aspiration (I/A) or vitrectomy handpiece. Also, the ultrasonic transmitter may be replaced by other means for emulsifying a lens, such as a high energy laser beam. The ultrasound energy from handpiece 12 helps to fragment the tissue of the lens, which can then be drawn into a port of the tip by aspiration flow. So as to balance the volume of material removed by the aspiration flow, an irrigation flow through handpiece 12 (or a separate probe structure) may also be provided, with both the aspiration and irrigations flows being controlled by console 14.

So as to avoid cross-contamination between patients without incurring excessive expenditures for each procedure, cassette 100 and its flexible conduit 18 may be disposable. Alternatively, the flexible conduit or tubing may be disposable, with the cassette body and/or other structures of the cassette being sterilizable. Regardless, the disposable components of the cassette are typically configured for use with a single patient, and may not be suitable for sterilization. The cassette will interface with reusable (and often quite expensive) components of console 14, which may include one or more peristaltic pump rollers, a Venturi or other vacuum source, a controller 40, and the like.

Controller 40 may include an embedded microcontroller and/or many of the components common to a personal computer, such as a processor, data bus, a memory, input and/or output devices (including a touch screen user interface 42), and the like. Controller 40 will often include both hardware and software, with the software typically comprising machine readable code or programming instructions for implementing one, some, or all of the methods described herein. The code may be embodied by a tangible media such as a memory, a magnetic recording media, an optical recording media, or the like. Controller 40 may have (or be coupled to) a recording media reader, or the code may be transmitted to controller 40 by a network connection such as an internet, an intranet, an Ethernet, a wireless network, or the like. Along with programming code, controller 40 may include stored data for implementing the methods described herein, and may generate and/or store data that records parameters corresponding to the treatment of one or more patients. Many components of console 14 may be found in or modified from known commercial phacoemulsification systems, such as those from Johnson & Johnson Surgical Vision, Inc. of Santa Ana, Calif.

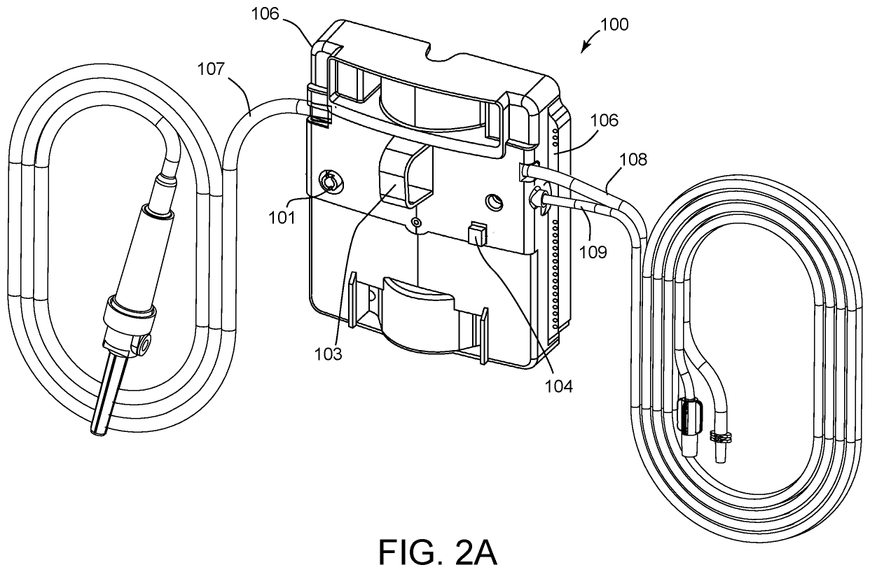

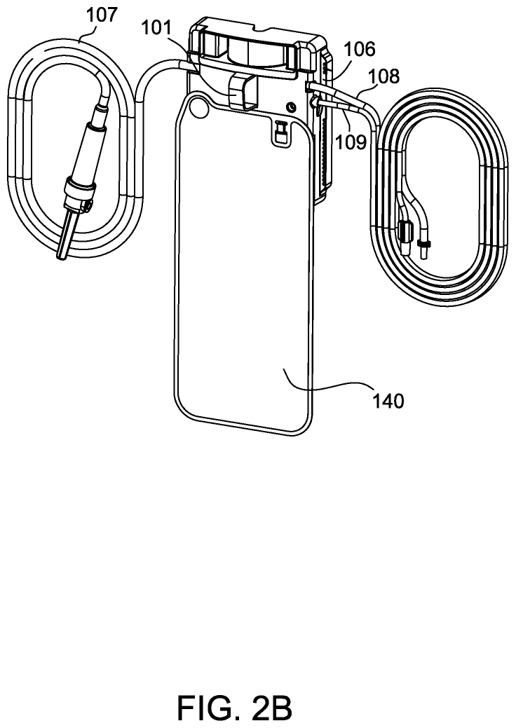

FIG. 2a illustrates a surgical cassette of the present invention, including components of surgical cassette 100. Surgical cassette 100 is an assembly of fluid pathways and connected tubing configured to manage one or more of the following: fluid inflow, fluid outflow, fluid vacuum level, and fluid pressure in a patient's eye E when coupled with console 14. Surgical cassette 100 may include grip loop handle 101, which provides a sterile means for holding and positioning surgical cassette 100 under finger grip control. In an embodiment, grip loop handle 101 is designed for an index finger to pass completely thru the loop of the handle. The grip loop handle 101 may also be designed for the pad of the thumb to rest on outer top surface of grip loop handle 101.

In an embodiment, surgical cassette 100 may include drain bag port 103. As illustrated in FIG. 2a, drain bag port 103 is an axially extending cylindrical port with a central opening to enable the transfer of fluid from the inside of the surgical cassette 100 manifold to an externally attached collection reservoir such as drain bag or collection vessel 140 (see FIG. 2b). As illustrated in FIG. 2a, surgical cassette 100 may include a drain bag hook 104. Drain bag hook 104 is a mechanical feature extending outward from the surface of surgical cassette 104 and is configured to interface with a corresponding slot feature in the drain bag 140 (see FIG. 2b) to support the weight of the drain bag as it collects fluid.

Surgical cassette 100 may also include one or more damping domes 106. As illustrated in FIG. 2a, clamping domes 106 may be a raised pattern of spherical domed surfaces with a single high-point to provide low friction wiping contact surfaces during loading and concentrate axial clamping forces in specific zones after loading surgical cassette 100 with console 14. It is also envisioned that the one or more clamping domes 106 may be of any shape or size suitable for its function or desired aesthetic look and feel.

In an embodiment, surgical cassette 100 may include irrigation inlet tubing port 107, irrigation outlet tubing 108, and aspiration outlet tubing 109. Irrigation inlet tubing port 107 may be a connection port for tubing extending to the balance salt solution (BSS) irrigation bottle to deliver irrigation fluid to fluid flow channels in surgical cassette 100 (not shown). irrigation outlet tubing 108 may extend to the surgical handpiece 12 to deliver irrigation fluid to patient's eye E. Aspiration outlet tubing 109 may extend to the surgical handpiece 12 for removing fluid from a patient's eye E by means of a pump, such as a flow-based pump, preferably a peristaltic pump, for example.

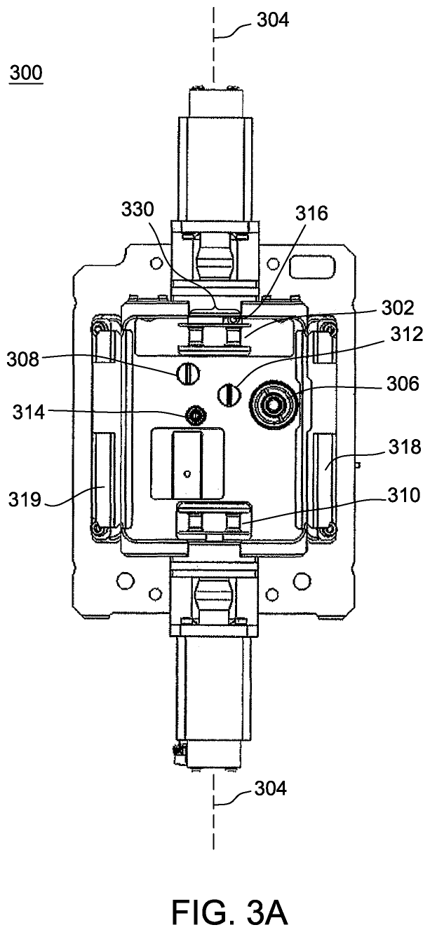

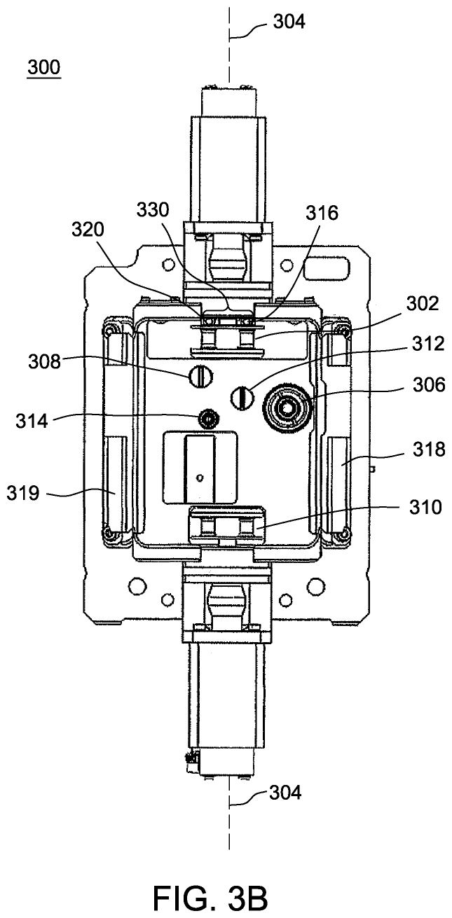

FIGS. 3A and 3B illustrate aspects of the surgical console of the present invention which may interchangeably receive multiple types of fluidic cassettes designs, for example, to be used with varying aspiration controls. Engagement between the cassette 100 and the cassette receptacle 300 of console 14 can be understood with reference to FIG. 3A. In particular, aspiration drive rotor 302 rotates about axis 304 and drives a peristaltic pump in the cassette 100. Pressure receiver 306 and valve actuator 308 respectively couple with a vacuum sensor and an irrigation valve mounted in either type of cassette. When a cassette 100 is mounted on the console 14, drain drive rotor 310 rotates about axis 304 to drive a peristaltic drain pump in the cassette 100. Valve actuator 312 may be coupled with a switching valve of cassette 100 and vacuum coupler 314 may couple with a holding tank associated with cassette 100. A cassette loaded into cassette receptacle 300 may actuate a cassette pre-load detection system 330 having detection switch 316 within cassette receptacle 300 and may provide an indication to the console of what type of cassette is present. It should be understood that the console may use other methods to actively detect or passively receive information from the mounted cassette regarding its functionality.

Detection switch 316 may be a spring-loaded pin displaced rearwards when surgical cassette 100 is initially inserted with an end or side surface triggering a switch and initiating closure of damps 318, 319. Detection switch 316 may be a switch component that changes electrical output state when cassette displaced to a specific axial position indicating surgical cassette 100 is in an appropriate position for loading engagement by clamps 318, 319, for example, and may identify the surgical cassette 100 and confirm functionality and/or compatibility with the console 14.

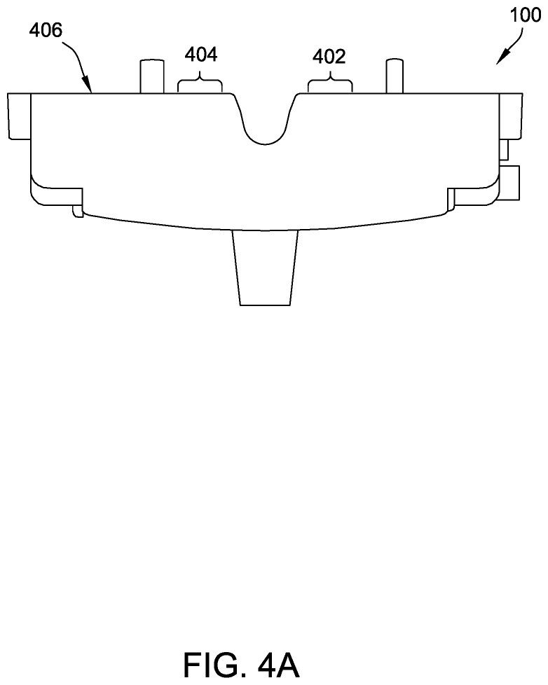

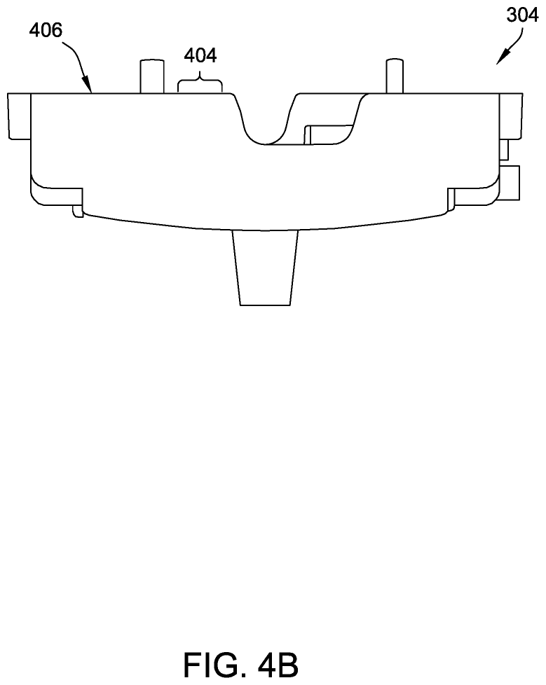

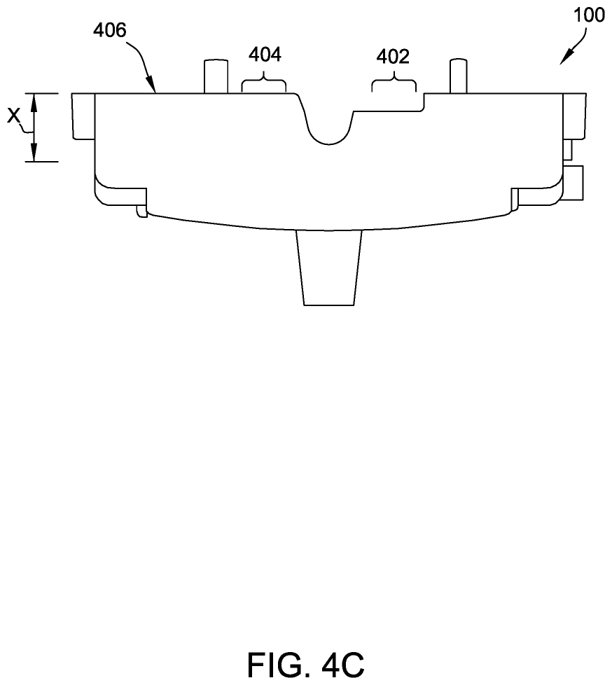

In an embodiment of the invention, console 14 may be configured to receive various types of cassettes. To this end, console 14 and the cassette may communicate to establish the functionality of the mounted cassette (i.e., the modes of aspiration enabled by the cassette). In one approach, a cassette may include a body portion that may be detected by the console and which identifies the available functionalities of the installed cassette. For example, with reference to FIGS. 3A and 4A, cassette 100 may include body portions 402 and 404; and in an embodiment, portion 402 may actuate cassette pre-load detection system 330 having detection switch 316 when cassette 100 is loaded into cassette receptacle 300 and indicate to the console that a first type of cassette has been installed. In response, the console 14 may, for example, use system settings associated for the inserted cassette, e.g. optimal vacuum, aspiration, and/or irrigation settings, and/or activate various components of the system, e.g. vacuum pump, pressurized irrigation, and other necessary mechanism in preparation for the surgical procedure.

Conversely, if the detection switch 316 is not triggered, the console may be informed that a second type of cassette 100 is loaded into cassette receptacle 300. In an embodiment of the present invention, with reference to FIGS. 3A and 4B, cassette 100 may include only body portion 404 and may not actuate cassette pre-load detection system 330 having detection switch 316 when cassette 100 is loaded into cassette receptacle 300, indicating to the console that a second type of cassette is being installed. In response, the console 14 may, for example, fail to fully capture and install cassette 100 or may provide an indication to the console 14 of the identity of the cassette 100. Therefore, utilizing a functional indicator, the surgical console 14 may be informed upon mounting of a cassette which type of cassette is being loaded and if, for example, the cassette is compatible with console 14. Moreover, if the cassette is compatible, the function indicator enables the system to use the system settings associated for the inserted cassette, e.g. optimal or desired vacuum, aspiration, and/or irrigation settings, and/or activate various components of the system

In an embodiment of the invention, with reference to FIGS. 3B and 4B, cassette 100 may include only body portion 404 which may actuate cassette pre-load detection system 330 having additional detection switch 320 when cassette 100 is loaded into cassette receptacle 300 and indicate to the console that a second type of cassette has been installed. In response, the console 14 may, for example, use system settings associated for the inserted cassette, e.g. optimal vacuum, aspiration, and/or irrigation settings, and/or activate various components of the system. Conversely, if both detection switches 316, 320 of cassette pre-load detection system 330 are triggered, the console may be informed that a first type of cassette 100 is being loaded into cassette receptacle 300. In response, the console 14 may, for example, fail to fully capture and install the first type cassette 100 or may provide an indication to the console 14 of the identity of the cassette 100.

In an embodiment of the present invention, body portions 404, 402 of cassette 100 may be of differing depths from back plane 406, which defines the leading edge of the cassette body as positioned towards the cassette receptacle 300. As illustrated in FIG. 4C, body portion 402 may be of a smaller depth than body portion 404, for example. A cassette loaded into cassette receptacle 300 having body portions of varying depths may actuate the cassette pre-load detection system 330 differently than a cassette having body portions equally aligned with back plane 406. For example, detection switch 316 within cassette receptacle 300 may detect a different body portion depth than detection switch 320 and may provide an indication to console 14 allowing for identification of the inserted cassette 100. The depth of body portions 404, 402 may be of any depth and may preferably fall within distance X as illustrated in FIG. 4C. Using varying depths of body portions 404, 402 may allow for the identification of any number of cassettes which may each comprise a variety of required settings and use parameters.

In an embodiment of the present invention, an indication of cassette type may prompt controller 40 to provide a message to the user of console 14 through user interface 42 which may include, for example, an indicator that the cassette type is not compatible with the console or which system attributes have been selected by the console 14 to be compatible with the cassette 100. For example, a first type cassette may be compatible with a dual pump eye treatment system while a second type cassette may be compatible with a dual pump eye treatment system that also provides for pressurized infusion. Similarly, various cassette types may require certain settings (e.g. vacuum, aspiration, flow, irrigation, etc.) for optimum performance due to physical characteristics of the components of the cassette, e.g. tubing material(s) and compliance, inner and/outer diameter of the tubing, wall thickness of the tubing, length of the tubing, etc. The physical characteristics between cassettes types may differ, but still operate with the same console or some cassettes may be incompatible. The present invention permits cassette identification and system configuration to ensure proper cassette usage and settings.

All references cited herein are hereby incorporated by reference in their entirety including any references cited therein.

Although the present invention has been described in terms of specific embodiments, changes and modifications can be carried out without departing from the scope of the invention which is intended to be limited only by the scope of the claims.

We claim:

1. An eye surgical system, comprising:
a cassette having at least two body portions; and
a console, wherein the console comprises a cassette receptacle configured to couple the cassette with the console, wherein the cassette receptacle comprises a cassette preload detection system;
wherein the body portions of the cassette are configured to contact the cassette pre-load detection system, and wherein the configuration of the body portions identifies a type of cassette coupled with the console.

2. The system of claim 1, wherein the cassette pre-load detection system comprises two detection switches.

3. The system of claim 2, wherein the two detection switches are proximate to each other.

4. The system of claim 1, wherein each type of cassette corresponds to one or more console compliant attributes selected from the group consisting of tubing diameter, tubing material, tubing thickness, tubing length, vacuum setting, aspiration setting, fluid flow rate, and irrigation rate.

5. The system of claim 2, wherein the body portion is configured to contact one of the two detection switches proximate to a top of the cassette body.

6. The system of claim 2, wherein the detection switches comprise a microswitch, piezometer, or an optical switch.

7. The system of claim 1, wherein the console is configured to receive an indication of the type of cassette from a depth of one of the body portions contacting one of the detection switches.

8. The system of claim 1, wherein the console is configured to receive an indication of the type of cassette from the body portion contacting one of the detection switches.

9. The system of claim 1, wherein the console is configured to operate in accordance with the cassette type identified.

10. A surgical console configured to receive multiple types of eye treatment cassettes, comprising:
a cassette receptacle for interchangeably receiving multiple types of cassettes, wherein the console operates with a first type of cassette and does not operate with a second type of cassette; and
a console controller for receiving an indication of the type of cassette received by the cassette receptacle and controlling the operation of the console.

11. The surgical console of claim 10, wherein the cassette type indication is generated by a cassette pre-load detection system.

12. The system of claim 10, wherein the indication is received from a detection switch.

13. The system of claim 10, wherein the operation of the console corresponds to one or more console compliant attributes selected from the group consisting of tubing diameter, tubing material, tubing thickness, tubing length, vacuum setting, aspiration setting, fluid flow rate, and irrigation rate.

14. The system of claim 10, wherein the indication is generated by a detection switch in contact with a body portion of the cassette.

15. The system of claim 12, wherein the detection switch is a microswitch, piezometer, or an optical switch.

16. The system of claim 10, wherein the indication is indicative of the depth of a body portion of the first type of cassette or second type of cassette.

17. The system of claim 10, wherein the first type of cassette includes a cassette operable with the surgical console.

18. A method for identifying a type of eye treatment cassette, comprising:
receiving an eye treatment cassette in a cassette receptacle of a surgical console;
receiving an indication of the type of cassette from a cassette pre-load detection system having at least two detection switches; and
enabling operation of the console based on the indication of the type of cassette.

19. The method of claim 18, wherein the at least two detection switches are proximate to each other.

20. The method of claim 18, wherein the type of cassette corresponds to one or more console compliant attributes selected from the group consisting of tubing diameter, tubing material, tubing thickness, tubing length, vacuum setting, aspiration setting, fluid flow rate, and irrigation rate.

21. The method of claim 18, wherein a body portion of the cassette is configured to contact one of the at least two detection switches.

22. The method of claim 18, wherein a body portion of the cassette is configured to contact two of the at least two detection switches.

23. The method of claim 18, wherein the detection switches comprise a microswitch, piezometer, or an optical switch.

24. The method of claim 18, wherein the console receives the indication of the type of cassette from a depth of a body portion of the cassette contacting one of the at least two detection switches.

25. The method of claim 18, wherein the console receives the indication of the type of cassette from a body portion of the cassette contacting one of the two detection switches.

26. The method of claim 18, wherein the console operates in accordance with the cassette type based on the received indication.
